(19) United States
(12) Patent Application Publication
(10) Pub. No.: US 2019/0274377 A1
(43) Pub. Date: Sep. 12, 2019

(54) COOLING VEST

(71) Applicant: Fan Gao Le Trade (Shenzhen) Co.,Ltd, SHENZHEN (CN)

(72) Inventor: Wenyong YUE, SHENZHEN (CN)

(73) Assignee: Fan Gao Le Trade (Shenzhen) Co.,Ltd

(21) Appl. No.: 15/913,890

(22) Filed: Mar. 6, 2018

(51) Int. Cl. A41D 13/005; A62B 17/00; F25D 3/08

(52) U.S. Cl. CPC A41D 13/0058; A62B 17/005; F25D 3/08; A61F 2007/0019; F25D 2400/28; F25D 2400/40; F25D 2400/26

(57) ABSTRACT

A cooling vest is provided which comprises: a vest having an interior volume, and a left and right pocket attached on the front of the vest; an elongated polyurethane tube having a first and a second end, wherein the tube is arranged in the interior volume in a “U” shape; a pump; an ice bag having an ice box received therein, wherein the ice box is full of ice, the first end of the tube is connected with an upper inlet of the ice bag, a low outlet of the ice bag is connected with the pump, the second end of the tube is connected with the pump; a switch for switching on/off of the pump, wherein the ice bag, the pump and the switch are received in one pocket together; and a portable power electrically connected with the switch, wherein the portable power is received in the other pocket.

### TECHNICAL FIELD

The present invention relates to a cooling garment, and more specifically to an improved cooling vest with a cooling loop

### BACKGROUND OF THE PRESENT INVENTION

Exposure to abnormal or prolonged amounts of heat and humidity without relief or adequate fluid intake can cause various types of heat-related illness. The cooling vest can be used to cool the body of a healthy person during indoor or outdoor recreation, e.g. welding operations and baking paint etc., or while working in hot conditions, for example in boiler workshop, shipyard, steel plant, glass factory, power plant, injection molding factory, forging workshop etc., or a sick person to reduce his abnormal body temperature. The cooling vest can be used to protect those exposed to abnormal or prolonged amounts of heat, e.g. frontier guard, railway worker, sanitation worker, courier, hardhat builder, delivery driver, airline ground staff etc. The cooling vest is also widely used during outdoor exercises, such as hike, riding on a bicycle, climbing, caving, fishing etc.

Various types of body cooling devices are well known. However, none of the references provided has a vest that cools the body via cooling loop filled with water or other liquid and is portable with a pump and power bank.

Specifically, U.S. Published Patent Application Number 2012/0260398 to Stapleton discloses a vest having an outer layer and an inner layer, each of the layers in communication with an air plenum. The two air plenums are in communication so as to allow air to flow through the vest, whereby the air flow can help cool the wearer.

U.S. Pat. No. 6,189,327 to Strauss discloses a personal cooler that comprises a band that can be worn around a user's neck, head, or other portions of the body. The band comprises at least one heat dissipating member that is adapted to absorb heat from the user's body, and a liquid-retainable material with at least one air plenum for delivering cool air to the user.

U.S. Pat. No. 8,443,463 to Paull discloses a garment that is composed of a wicking fabric and includes a tube imbedded therein. The tube is in fluid communication with a reservoir that is removably attached thereto. The tube is preferably disposed along the top portion of the garment so as to dispense water or liquid to the top portion thereof.

The devices disclosed in the prior art have several known drawbacks. These devices are limited in that none of these devices are provided with a left and a right pocket for respectively receiving the pump and the portable power. None of these devices disclose that the fluid or water is stored in an ice box which is received in an ice bag, and the ice bag and the pump are received in one pocket together.

### SUMMARY OF PRESENT INVENTION

For the defects in the prior art, an object of the present invention is to provide a new and improved cooling vest wherein the same can be utilized for reducing the body temperature of a healthy person during indoor or outdoor recreation or while working in hot conditions, or a sick person.

The technical solutions of the present invention are as follows:

A cooling vest is provided, which comprises: a vest having an outer layer and an inner layer that are attached together about the edges thereof to form an interior volume therebetween, and a left and right pocket attached on the front of the vest; an elongated polyurethane tube having a first and a second end, wherein the tube is arranged in the interior volume in a “U” shape; a pump; an ice bag having an ice box received therein, wherein the ice box is full of ice, circulating water is filled between the ice bag and the ice box, the first end of the tube is connected with an upper inlet of the ice bag, a low outlet of the ice bag is connected with the pump, the second end of the tube is connected with the pump; a switch for switching on/off of the pump, wherein the ice bag, the pump and the switch are received in one pocket together; and a portable power electrically connected with the switch, wherein the portable power is received in the other pocket.

Advantageously, the pump and the ice bag are detachable from the vest and are capable of being taken out of the pocket.

Advantageously, the tube is arranged in the front, back and neck portion of the vest.

Advantageously, the portable power is connected to the vest via a USB connection.

Advantageously, the cooling vest further comprises a handbag as an accessory of the cooling vest for receiving a plurality of ice boxes.

Other objects, features, and advantages of the present invention will become apparent from the following detailed description taken in conjunction with the accompanying drawings.

### DESCRIPTION OF THE DRAWINGS

FIG. 1 shows a transparent front view of a cooling vest;

FIG. 2 shows a transparent back view of the cooling vest;

FIG. 3 shows a bottom view of the cooling vest;

FIG. 4 shows a schematic view of a handbag.

### DETAILED DESCRIPTION OF PREFERRED EMBODIMENTS

The present invention will now be more particularly described, by way of example only, with reference to the accompanying drawings. It should be understood that the drawing are for better understanding and should not limit the present invention. Dimensions of components and features shown in the drawings are generally chosen for convenience and clarity of presentation and are not necessarily shown to scale.

This disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

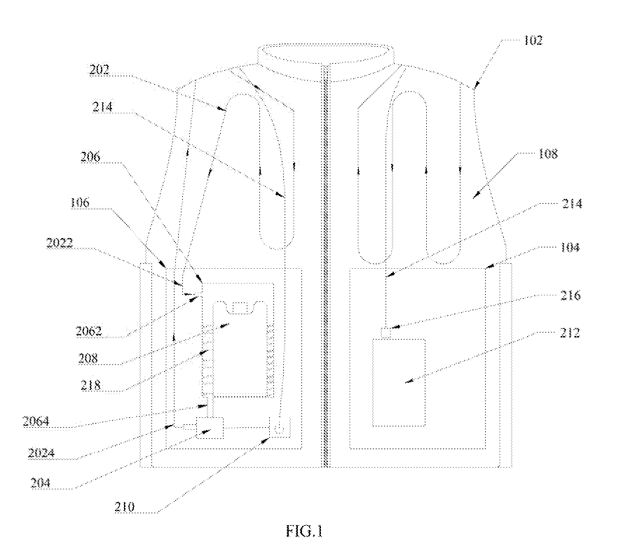

FIG. 1 shows an exemplary embodiment of transparent front view of a cooling vest comprising a vest 102 and a cooling system. The cooling system comprises an elongated polyurethane tube 202 having a first and a second end 2022 and 2024, a pump 204, an ice bag 206 having an ice box 208 received therein, a switch 210 for switching on/off of the pump 204 and a portable power 212 electrically connected with the switch 210 via a wire 214.

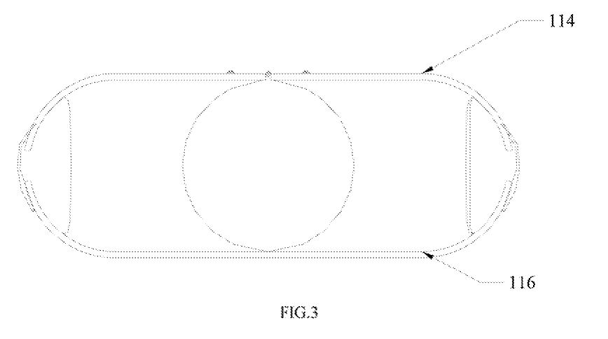

FIG. 3 shows a bottom view of the cooling vest. The vest 102 has an outer layer 114 and an inner layer 116 that are attached together about the edges thereof to form an interior volume therebetween, and a left and right pocket 104 and 106 attached on the front 108 of the vest 102.

As shown in FIG. 1, the tube 202 is arranged in the interior volume in a “U” shape. The ice box 208 is full of ice, and circulating water 218 is filled between the ice bag 206 and the ice box 208. The first end 2022 of the tube 202 is connected with an upper inlet 2062 of the ice bag 206, a low outlet 2064 of the ice bag 206 is connected with the pump 204, and the second end 2024 of the tube is connected with the pump 204. The ice bag 206 with the ice box 208, the pump 204 and the switch 210 are received in one pocket together, for example the right pocket 106, and the portable power 212 is received in the other pocket, for example the left pocket 104.

When working, the ice bag 206 and the pump 204 are connected to the tube 202, circulating water 218 is poured into the ice bag 206, the ice box 208 full of ice is put into the ice bag 206, and then the ice bag 206 is sealed. After closing the switch 210, the pump 204 is started and the cooling water in the ice bag 206 is pumped into the tube 202 that is arranged all around the vest 102 so as to cool user' body.

As shown in FIG. 1, the pump 204 and the ice bag 206 with the ice box 208 are detachable from the vest 102 and are capable of being taken out of the pocket 106. Preferably, the portable power 212 is connected to the wire 214 in the vest 102 via a USB connection 216.

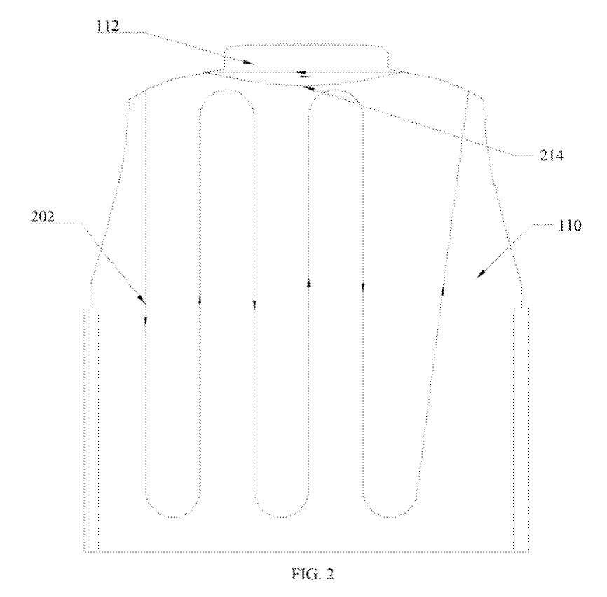

FIG. 2 shows a back view of the cooling vest. As shown in FIGS. 1 and 2, the tube 202 is arranged in the front 108, back 110 and neck 112 portion of the vest 102. Thus, the cooling effect can be improved significantly.

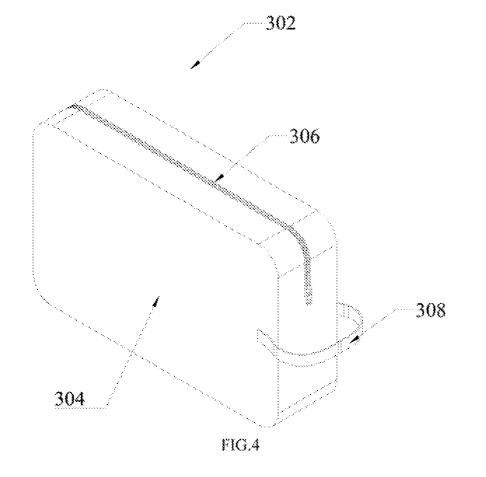

FIG. 4 shows a schematic view of a handbag 302 which functions as an accessory of the cooling vest. A plurality of ice boxes 208, e.g. 6 ice boxes, can be received in the handbag 302. However, the handbag 302 can be designed and sized to receive several ice boxes according to actual needs. As shown in FIG. 4, the handbag 302 comprises a bag body 304 having an interior volume for receiving the ice boxes 208, a zipper 306 arranged on a first side surface of the bag body 302, and a handle 308 for lifting arranged on a second side surface adjacent to the first side surface. The handbag 302 may further comprise a heat insulating layer arranged in the interior volume for protecting the ice boxes 208 from heat absorption. With the handbag 302, the cooling vest can last for a longer time during each use.

The complete assembly may be fabricated with cotton cloth or other fabric on the inside of the cooling vest and thermal cloth on the outer side. Between said cloth or fabric will have rubber-tubing in a closed circuit coil filled with water or other liquids that will be cooled to a desired temperature via a tube submersed in a cooler with ice and circulated via a pump. Furthermore the pump will be may have the ability to function via AC, DC or solar power current. With the invention of the cooling vest, many traumatic medical situations that occur after the initial trauma will be limited and/or prevented, thus enabling for a better recovery with much less negative after effects.

Although the invention is described with reference to one or more preferred embodiments, it should be appreciated by those skilled in the art that various modifications are possible. Therefore, the scope of the invention is to be determined by reference to the claims that follow.

I claim:

1. A cooling vest, comprising:
a vest having an outer layer and an inner layer that are attached together about the edges thereof to form an interior volume therebetween, and a left and right pocket attached on the front of the vest;
an elongated polyurethane tube having a first and a second end, wherein the tube is arranged in the interior volume in a “U” shape;
a pump;
an ice bag having an ice box received therein, wherein the ice box is full of ice, circulating water is filled between the ice bag and the ice box, the first end of the tube is connected with an upper inlet of the ice bag, a low outlet of the ice bag is connected with the pump, the second end of the tube is connected with the pump;
a switch for switching on/off of the pump, wherein the ice bag, the pump and the switch are received in one pocket together; and
a portable power electrically connected with the switch, wherein the portable power is received in the other pocket.

2. The cooling vest according to claim 1, wherein the pump and the ice bag are detachable from the vest and are capable of being taken out of the pocket.

3. The cooling vest according to claim 1, wherein the tube is arranged in the front, back and neck portion of the vest.

4. The cooling vest according to claim 1, wherein the portable power is connected to the vest via a USB connection.

5. The cooling vest according to claim 1, further comprising a handbag as an accessory of the cooling vest for receiving a plurality of ice boxes.
